(19) United States
(12) Patent Application Publication
(10) Pub. No.: US 2022/0387654 A1
(43) Pub. Date: Dec. 8, 2022

(54) DEVICE FOR THE DIFFUSION OF VOLATILE SUBSTANCES

(71) Applicant: ZOBELE HOLDING SPA, Trento (IT)

(72) Inventors: Dominic DOYLE, Barcelona (ES); Roberto CAMARERO DÍEZ, Barcelona (ES); Fernando ALFONSO GALLEGO, Barcelona (ES)

(73) Assignee: ZOBELE HOLDING SPA, Trento (IT)

(21) Appl. No.: 17/789,261

(22) PCT Filed: Dec. 28, 2020
(86) PCT No.: PCT/EP2020/087915

§ 371 Date: Jun. 27, 2022

(30) Foreign Application Priority Data
Dec. 27, 2019 (ES) P201931158

(51) Int. Cl. A61L 9/12; A61L 9/04

(52) U.S. Cl. CPC A61L 9/127; A61L 9/042

(57) ABSTRACT

The device for the diffusion of volatile substances comprises a container (1), which holds a liquid with the volatile substances in it, and which has an opening (9), for communication from the inside of the housing (6) to the outside; a cover (2) which can be attached to the container (1) to close the opening (9), a diffusion wick (4) for the controlled diffusion of the volatile substances, one end (13) of which protrudes from the cover (2); and a cap (5) which covers the end (13) of the diffusion wick (4) which protrudes from the cover (2), the container (1), the cover (2), the diffusion wick (4) and the cap (5) being made of the same plastic material. It thus makes it possible to provide more efficient recyclability in accordance with currently existing methods.

### OBJECT OF THE INVENTION

The present invention falls within the technical field of air disinfection, sterilisation or deodorisation apparatus, more specifically those using substances that can be evaporated into the air, and concerns in particular a device for the diffusion of volatile substances, in which all its constituent elements are made of the same plastic material.

This feature of the device represents an advantage in terms of environmental sustainability, since the container, once discarded, does not need to be dismantled for efficient separation and recycling of each of its constituent elements. As these are made of the same and unique plastic material, the end user can deposit the used device directly into a plastic container, of those normally used for separating recyclable waste.

### BACKGROUND TO THE INVENTION

Within the technical field of diffusion devices for volatile substances, a first distinction should be made between those made of glass type materials and those made of plastics.

Glass containers containing volatile substances, such as liquid fragrances or insecticide compositions, can be recycled for the most part. However, these containers necessarily incorporate capillary type elements to allow the diffusion of the liquid contained to the external environment. These diffusing elements are usually linked to the body of the container in such a way as to ensure that the liquid is not accessible to a user, resulting in a container composed of at least two very different materials, which are difficult to separate from each other and therefore impossible to recycle in a proper way, thus contaminating the final recycled material or being simply disposed of in landfills.

The components of the plastic containers can also be recycled for the most part. However, similarly to glass containers, their final assembly is the result of a mixture of different plastics, which normally cannot be easily separated at the time of disposal.

Even if the components can be dismantled, it is a nuisance for the end user, which discourages proper separation and recycling.

Various patent documents relating to containers for volatile releasable substances are known in the current state of the art, each of which has a wide variety of advantageous characteristics and functionalities.

Most devices for the diffusion of volatile substances differ from each other in a variety of functionalities, without special attention having been paid so far to the recyclability of their components.

There is therefore a need for devices for the diffusion of volatile substances which allow for the separation and recycling of their constituent elements in an effective and simple manner. It is also desirable that such containers should meet the necessary conditions of safety and tightness, and that they should be of simple and economical construction.

### DESCRIPTION OF THE INVENTION

The object of the invention consists of a device for the diffusion of volatile substances in accordance with claim 1, in particular, a device for the diffusion of volatile substances comprising a porous and capillary element allowing the substances to be diffused into the surrounding air. All the materials making up the device are made of a single type of plastic, thus providing more efficient recyclability in accordance with currently existing methods. Thus, once the liquid contained in the container has been consumed, the user does not need to dismantle it to separate its components for recycling, but simply places it in one of the containers provided for this purpose.

The device comprises a main container for housing volatile substances (whether liquid fragrances or insecticide compositions), to which a closing lid is attached, equipped with a slot through which a diffusing wick is passed. The lid is covered by a cap that prevents the diffusion of the impregnated wick during transport, storage or any period when the product is not in use.

Other characteristics options are defined in the dependent claims.

In a first preferred embodiment, the components of the device are materialized in polypropylene (PP), while in a second preferred embodiment, the plastic used for the materialization of these components is polyethylene (PE).

The main novelty of the container is that the wick material, which is the aforementioned porous and capillary element that allows the substance to diffuse into the surrounding air, is made of a plastic material similar to that of the other components, thus facilitating its recycling. The plastic used for the wick ensures the necessary functional properties of capillary diffusion.

On the other hand, the groove through which the wick passes usually also includes an adjustment and sealing element, which helps to prevent the liquid contained in the container from spilling through the groove, and also serves to hold the wick in the groove to prevent it from being removed and possible leaks. This adjustment element, due to the functional properties it must have (certain elasticity and flexibility, adjustment capacity), is usually made of plastics that are very different from those used in the rest of the device.

Since the wick of the device described here can be produced by plastic moulding in a three-dimensional shape, or by cutting in a two-dimensional shape, the presence of the above-mentioned adjustment and sealing element is not necessary, as its perfect fit can be ensured by the grooving of the closing cap. In this way, the design and use of materials in the device can be simplified, contributing to easier and more homogeneous recycling.

The device for diffusing volatile substances described above can be connected to an external diffusion device or can be used independently, as it has its own diffusion media.

### DESCRIPTION OF THE DRAWINGS

To complement the description being made and in order to help a better understanding of the characteristics of the invention, in accordance with a preferential example of practical implementation of the same, is accompanied as an integral part of that description a set of drawings where, for illustrative purposes and not limited, the following has been represented:

FIG. 1.—Shows a perspective view of the device for diffusion of volatile substances according to the present invention.

FIG. 2.—Shows an exploded view of the device, in which its main constituent elements can be seen.

FIG. 3.1.—Shows a front view of the device.

FIG. 3.2.—Shows a first side view of the device.

FIG. 3.3.—Shows a side view of the device, sectioned according to the imaginary line III-III in FIG. 3.1.

FIG. 4.1.—Shows a second side view of the device.

FIG. 4.2.—Shows a front view of the device, sectioned according to the imaginary line IV-IV in FIG. 4.1.

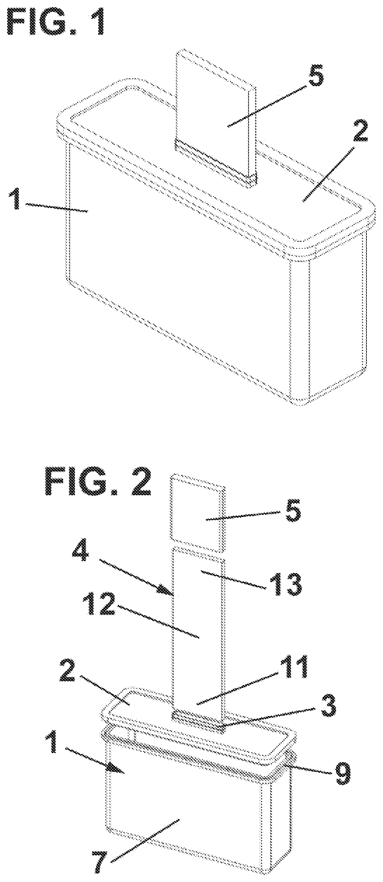

### PREFERRED EMBODIMENT OF THE INVENTION

A detailed explanation of an example of a preferred embodiment of the object of the present invention is given below with the help of the above-mentioned figures.

The device for diffusing volatile substances described, shown in the enclosed figures, comprises a container (1), a cover (2) for closing the container (1), equipped with a slot (3), and a diffusion wick (4) that can be fitted into the slot (3), which is partially covered by a cap (5).

The container (1) is made up of a hollow prismatic body, which has an interior housing (6) delimited by lateral walls (7) and a closed lower base (8), and an upper opening (9). The housing (6) is sized and configured to contain a volume of a volatile substance, usually in liquid form and consisting of a fragrance or an insecticide composition. This container (1) is made of a plastic material, which in this case is polypropylene.

The lid (2) of the container (1) has a geometry and dimensions corresponding to those of the opening (9) which it covers, as well as including couplings (10) for physical coupling with the body of the container (1). As indicated above, this cover (2), which is made of a plastic material, preferably polypropylene, has a longitudinal slot (3) for connecting the inner housing (6) with the outer medium to the device.

The diffuser wick (4) can be fitted transversely in the groove (3) to allow controlled release of the volatile substance from inside the housing (6). To this end, the diffuser wick (4) is made up of a body of porous plastic material that allows capillary flow through it. In this preferred embodiment, the body is laminated and has a rectangular geometry, and the plastic in which it is made is sintered polypropylene.

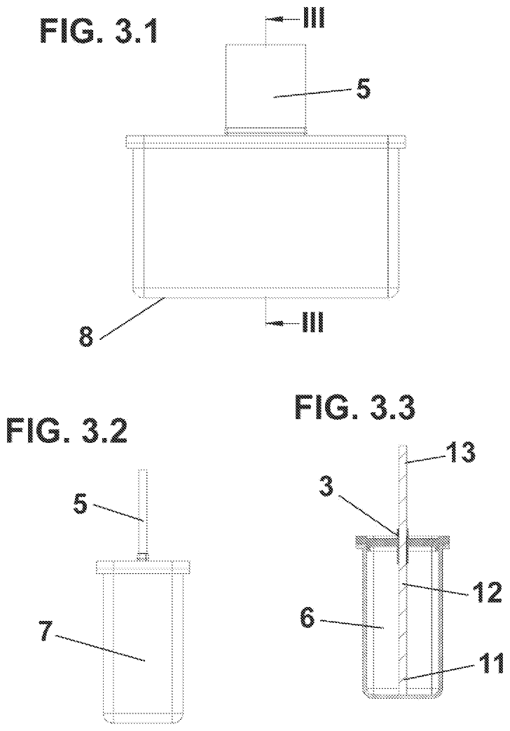

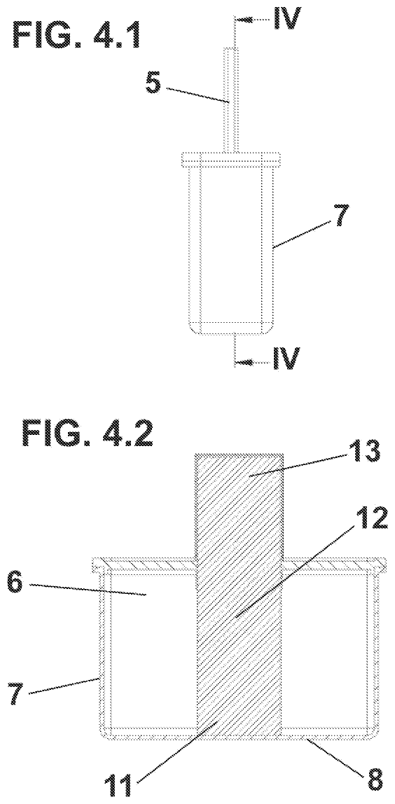

Thus, the diffuser wick (4) has a first end (11), which can be turned towards the housing (6), a central sector (12) which can be fitted into the slot (3), and a second end (13), which can be turned outwards. The volatile substance inside the housing (6) impregnates the first end (11) of the diffuser wick (4), with which it is in direct contact, and flows upwards by capillary action to the central sector (12) and finally to the second end (13), through which it is released by volatilisation into the surrounding environment, as can be seen in FIGS. 3.3 and 4.2.

The cap (5) consists of a plastic part, preferably made of polypropylene, configured and sized for coupling and covering the second end (13) of the diffuser wick (4), to prevent the release of the volatile substance by volatilisation during transport and storage of the device, or during any period when it is desired to prevent such release.

In the first preferred embodiment that is being described, in which all the components are made of polypropylene, they are assembled solidly and mutually by means of ultrasonic welding.

In a second preferred embodiment, in which the assembly between the components of the device is carried out by physical means, this device incorporates at least one adjustment and sealing element, not shown in the attached figures, which ensures correct coupling of the diffuser wick (4) in the slot (3) and prevents possible leaks and spillage of the contents of the housing (6) of the container (1).

An alternative design is also envisaged in which the diffuser wick itself (4) incorporates additional elements for coupling with the groove (3), thus avoiding the need for the adjustment and sealing element. In this alternative embodiment, in which the diffuser wick (4) is moulded, the additional coupling elements consist of a defined extension in the central sector (12) for locking and sealing inside the slot (3).

1. Device for the diffusion of volatile substances, comprising:
a container (1), which holds a liquid with the volatile substances, and which includes an opening (9), for communication from the inside of the housing (6) to the outside;
a lid (2) that can be attached to the container (1) to close the opening (9);
a diffuser wick (4) for controlled diffusion of volatile substances, one end (13) of which protrudes from the cover (2); and
a cap (5) covering the end (13) of the diffuser wick (4) which protrudes from the cap (2);
characterised in that the container (1), the lid (2), the diffuser wick (4) and the cap (5) are all made of the same plastic material.

2. Device for the diffusion of volatile substances according to claim 1, wherein the plastic material is polypropylene

3. Device for the diffusion of volatile substances according to claim 1, wherein the plastic material is polyethylene.

4. Volatile substance diffusion device according to claim 1 wherein the cover (2) comprises a slot (3) through which the diffusion wick (4) is placed.

5. Diffusion device for volatile substances according to claim 1 wherein the diffusion wick (4) comprises a seal extension.

6. Diffusion device for volatile substances according to claim 5 wherein the seal extension is defined in a central sector (12) of the diffusion wick (4) that can be fitted into this slot (3).
